(19) United States
(12) Patent Application Publication
(10) Pub. No.: US 2024/0379246 A1
(43) Pub. Date: Nov. 14, 2024

(54) BEAT AND ECG DATA ANALYSIS PRIORITIZATION

(71) Applicant: Boston Scientific Cardiac Diagnostics, Inc., Rochester, MN (US)

(72) Inventors: Drew Lohmeyer, Rochester, MN (US); Jeffrey R. Spors, Eyota, MN (US); David R. Engebretsen, Cannon Falls, MN (US)

(21) Appl. No.: 18/655,774

(22) Filed: May 6, 2024

Related U.S. Application Data
(60) Provisional application No. 63/464,672, filed on May 8, 2023.

(51) Int. Cl. G16H 50/70; A61B 5/00; A61B 5/339; A61B 5/364; G16H 10/20; G16H 40/67

(52) U.S. Cl. CPC G16H 50/70; A61B 5/0006; A61B 5/339; A61B 5/364; A61B 5/7475; G16H 10/20; G16H 40/67

(57) ABSTRACT

Systems and methods involve selectively downloading electrocardiogram (ECG) data associated with a patient study session according to a dynamic priority protocol and displaying plots of the ECG data that have been downloaded. The dynamic priority protocol is structured to prioritize downloading strips of the ECG data associated with beat classifications indicating ectopic beats.

### CROSS REFERENCE TO RELATED APPLICATION

This application claims priority to Provisional Application No. 63/464,672, filed May 8, 2023, which is herein incorporated by reference in its entirety.

### TECHNICAL FIELD

The present disclosure relates to devices, methods, and systems for monitoring, identifying, and classifying cardiac events.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes receiving, at a remote computer, a selection of a patient study session; selectively downloading, to the remote computer, electrocardiogram (ECG) data associated with the patient study session according to a dynamic priority protocol; and displaying, on a user interface of the remote computer, plots of the ECG data that have been downloaded. The dynamic priority protocol is structured to prioritize downloading strips of the ECG data associated with beat classifications indicating ectopic beats.

In Example 2, the method of Example 1, wherein the beat classifications indicating ectopic beats are ventricular beats and supraventricular beats.

In Example 3, the method of Example 1, wherein the strips of ECG data containing at least one ventricular beat are prioritized first, wherein the strips of ECG data containing at least one supraventricular beat are prioritized second.

In Example 4, the method of Examples 2 or 3, wherein the displaying includes: displaying the plots of the ECG data associated with ventricular beats in a first window in the user interface and displaying the plots of the ECG data associated with supraventricular beats in a second window in the user interface.

In Example 5, the method of any of Examples 1-4, wherein the dynamic priority protocol is modified in response to detecting that the user is scrolling through sections of the user interface associated with ECG data that has not been downloaded.

In Example 6, the method of any of Examples 1-5, further including: before downloading the ECG data, downloading to the remote computer a first data payload comprising pointers to the ECG data.

In Example 7, the method of Example 6, further including: before downloading the ECG data and after downloading the first data payload, downloading to the remote computer a second data payload comprising the metadata indicating cardiac event classifications, the beat classifications, and locations of beats.

In Example 8, the method of any of Examples 1-7, further including: displaying on the user interface a first indicator representing an amount of the ECG data associated with a first beat classification that has been downloaded to the remote computer; and displaying on the user interface a second indicator representing an amount of the ECG data associated with a second beat classification that has been downloaded to the remote computer.

In Example 9, the method of Example 8, wherein the ECG data associated with the first beat classification is prioritized for downloading in response to a user selecting the first indicator.

In Example 10, the method of any of Examples 1-9, wherein the dynamic priority protocol includes a set of default rules.

In Example 11, the method of Example 10, wherein the default rules are modified by a user's activity on the user interface.

In Example 12, the method of any of Examples 1-11, wherein the strips of ECG data are 5- to 10-minute-long strips.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a system includes: a remote computing system with: a user interface, a first processor, and a first computer-readable medium having a first set of computer-executable instructions. The first set of computer-executable instructions are configured to be executed by the first processor to cause the remote computing system to: send requests to a server to retrieve electrocardiogram (ECG) data associated with a patient study session; receive the ECG data in response to the requests; and display, on the user interface, plots of the ECG data that have been received. The requests are generated according to a dynamic priority protocol. The ECG data is associated with metadata, which includes beat classifications. And the dynamic priority protocol prioritizes downloading strips of the ECG data associated with the beat classifications indicating ectopic beats.

In Example 17, the system of Example 16, wherein the beat classifications indicating ectopic beats are ventricular beats and supraventricular beats.

In Example 18, the system of Example 16, wherein the strips of ECG data containing at least one ventricular beat are prioritized first, wherein the strips of ECG data containing at least one supraventricular beat are prioritized second.

In Example 19, the system of Example 16, wherein the display of the plots of the ECG data includes: display the plots of the ECG data associated with ventricular beats in a first window in the user interface and displaying the plots of the ECG data associated with supraventricular beats in a second window in the user interface.

In Example 20, the system of Example 16, wherein the dynamic priority protocol is modified in response to detecting that a user is scrolling through sections of the user interface associated with ECG data that has not been downloaded.

In Example 21, the system of Example 16, wherein the first set of computer-executable instructions are configured to be executed by the first processor to cause the remote computing system to: display on the user interface a first indicator representing an amount of the ECG data associated with a first beat classification that has been downloaded to the remote computer; and display on the user interface a second indicator representing an amount of the ECG data associated with a second beat classification that has been downloaded to the remote computer.

In Example 22, the system of Example 21, wherein the ECG data associated with the first beat classification is prioritized for downloading in response to a user selecting the first indicator.

In Example 23, the system of Example 16, wherein the dynamic priority protocol includes a set of default rules.

In Example 24, the system of Example 23, wherein the default rules are modified by a user's activity on the user interface.

In Example 25, the system of Example 16, wherein the first set of instructions are part of the package of data received from a server.

In Example 26, the system of Example 25, wherein the first set of instructions are executed by a web browser at the remote computing system.

In Example 27, the system of Example 16, wherein the requests each include a pointer to respective strips of ECG data containing beat classifications indicating ectopic beats.

In Example 28, the system of Example 27, wherein the strips of ECG data are 5- to 10-minute-long strips.

In Example 29, the system of Example 16, wherein the server includes: a database, a second processor, and a second computer-readable medium having a second set of computer-executable instructions. The second set of computer-executable instructions are configured to be executed by the second processor to cause the server to: in response to the requests, retrieve the ECG data from the database and transmit the strips of the ECG data to the remote computing system.

In Example 30, a method includes receiving, at a remote computer, a selection of a patient study session; selectively downloading, to the remote computer, electrocardiogram (ECG) data associated with the patient study session according to a dynamic priority protocol; and displaying, on a user interface of the remote computer, plots of the ECG data that have been downloaded. The ECG data is associated with metadata that includes beat classifications, and the dynamic priority protocol prioritizes downloading strips of the ECG data associated with the beat classifications indicating ectopic beats.

In Example 31, the method of Example 30, wherein the beat classifications indicating ectopic beats are ventricular beats and supraventricular beats.

In Example 32, the method of Example 30, wherein the strips of ECG data containing at least one ventricular beat are prioritized first, wherein the strips of ECG data containing at least one supraventricular beat are prioritized second.

In Example 33, the method of Example 31, wherein the displaying includes: displaying the plots of the ECG data associated with ventricular beats in a first window in the user interface and displaying the plots of the ECG data associated with supraventricular beats in a second window in the user interface.

In Example 34, the method of Example 30, wherein the metadata with the beat classifications are downloaded to the remote computer before the ECG data is downloaded to the remote computer.

In Example 35, the method of Example 30, wherein the dynamic priority protocol includes a set of default rules, wherein the default rules are modified by a user's activity on the user interface.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, a remote computer, and a user interface, in accordance with certain instances of the present disclosure.

FIG. 3 shows an example of beats that have been grouped together and displayed in a user interface, in accordance with certain instances of the present disclosure.

FIG. 4 shows a block diagram depicting an illustrative method, in accordance with certain instances of the disclosure.

FIG. 5 shows a block diagram depicting a user interface, in accordance with certain instances of the disclosure.

FIG. 6 shows a user interface with windows displaying plots of ECG data of beats that have been grouped together, in accordance with certain instances of the present disclosure.

FIG. 7 is a block diagram depicting an illustrative computing device, in accordance with instances of the disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Electrocardiogram (ECG) data of a patient can be used to analyze a patient's cardiac activity and recommend treatments. To collect ECG data, one or more monitoring devices (e.g., sensors) can be coupled to the patient such that the monitoring devices sense and record the ECG data. The amount of ECG data can be overwhelming to store, process, and analyze locally by the monitoring devices or mobile devices. Providing access to large sets of ECG data (e.g., days of ECG data) can be challenging with respect to computing resources and network bandwidth. Instances of the present disclosure are accordingly directed to systems, methods, and devices for prioritizing access to ECG data and metadata to facilitate efficient analysis of cardiac activity.

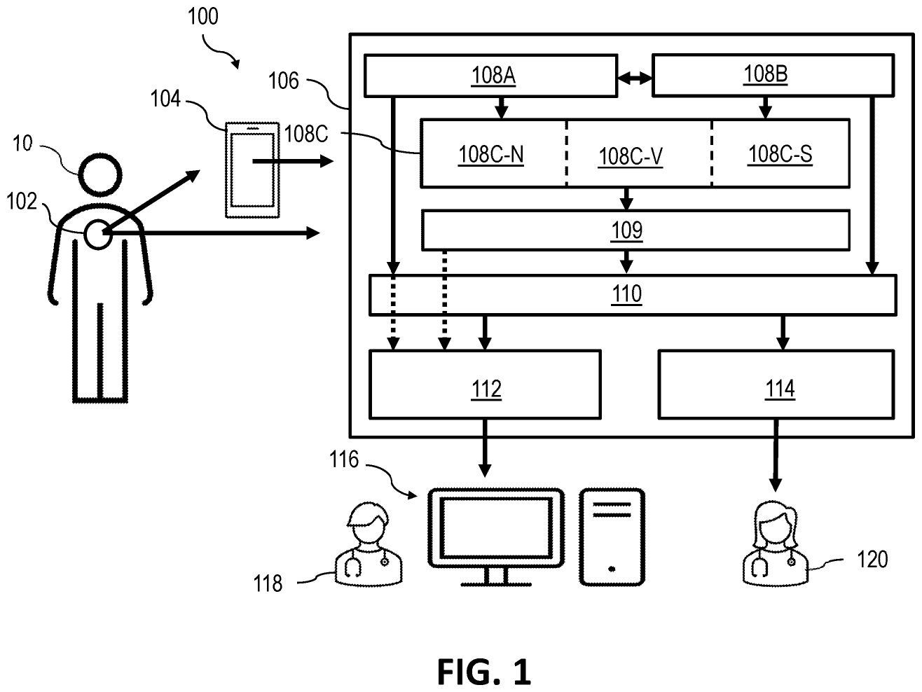

FIG. 1 illustrates a patient 10 and an example system 100. The system 100 includes a monitor 102 attached to the patient 10 or implanted in the patient 10 (e.g., pacemaker, ICD, CRT, or ICM) to detect cardiac activity of the patient 10. The monitor 102 may produce electric signals that represent the cardiac activity in the patient 10. For example, the monitor 102 may detect the patient's heart beating (e.g., using infrared sensors, electrodes, heart sounds) and convert the detected heartbeat into electric signals representing ECG data. In certain instances, the monitor 102 stores the ECG data of a patient study (e.g., one or more days of ECG data), after which the ECG data is transmitted to another device or system such as a server. Additionally or alternatively, the monitor 102 transmits the ECG data to a mobile device 104 (e.g., a mobile phone). In such instances, the mobile device 104 can include a program (e.g., mobile phone application) that receives, processes, and analyzes the ECG data. For example, the program may analyze the ECG data and detect or flag cardiac events (e.g., periods of irregular cardiac activity) contained within the ECG data.

The mobile device 104 can periodically transmit chunks of the ECG data to another device or system such as a server, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104. Also, the monitor 102 and/or the mobile device 104 includes a button or touch-screen icon that allows the patient 10 to initiate an event. Such an indication can be recorded and communicated to the other device or system. In other instances involving multi-day studies, the ECG data and associated metadata are transmitted in larger chunks (e.g., an entire study's worth of ECG data).

### Cardiac Event Server

The ECG data (and associated metadata, if any) is transmitted to and stored by a cardiac event server 106 (hereinafter “the server 106” for brevity). The server 106 includes multiple models, platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108A, 108B, and 108C, a clustering algorithm module 109, a cardiac event router 110, a report platform 112, and a notification platform 114. Although only one server 106 is shown in FIG. 1, the server 106 can include multiple separate physical servers, and the various models/platforms/modules/layers can be distributed among the multiple servers. Each of the models/platforms/modules/layers can represent separate programs, applications, and/or blocks of code where the output of one of the models/platforms/modules/layers is an input to another of the models/platforms/modules/layers. Each of the models/platforms/modules/layers can use application programming interfaces to communicate between or among the other models/platforms/modules/layers as well as systems and devices external to the server 106.

In certain instances, once the ECG data is processed by the machine learning models 108A-C and the clustering algorithm module 109, the ECG data (and associated metadata) is made available for the report platform 112. As will be described in more detail below, the report platform 112 can be accessed by a remote computer 116 (e.g., client device such as a laptop, mobile phone, desktop computer, and the like) by a user at a clinic or lab 118. In other instances, the cardiac event router 110 is used to determine what platform further processes the ECG data based on the classification associated with the cardiac event. For example, if the identified cardiac event is critical or severe, the cardiac event router 110 can flag or send the ECG data, etc., to the notification platform 114. The notification platform 114 can be programmed to send notifications (along with relevant ECG data and associated metadata) immediately to the patient's physician/care group remote computer 116 and/or to the patient 10 (e.g., to their computer system, e-mail, mobile phone application).

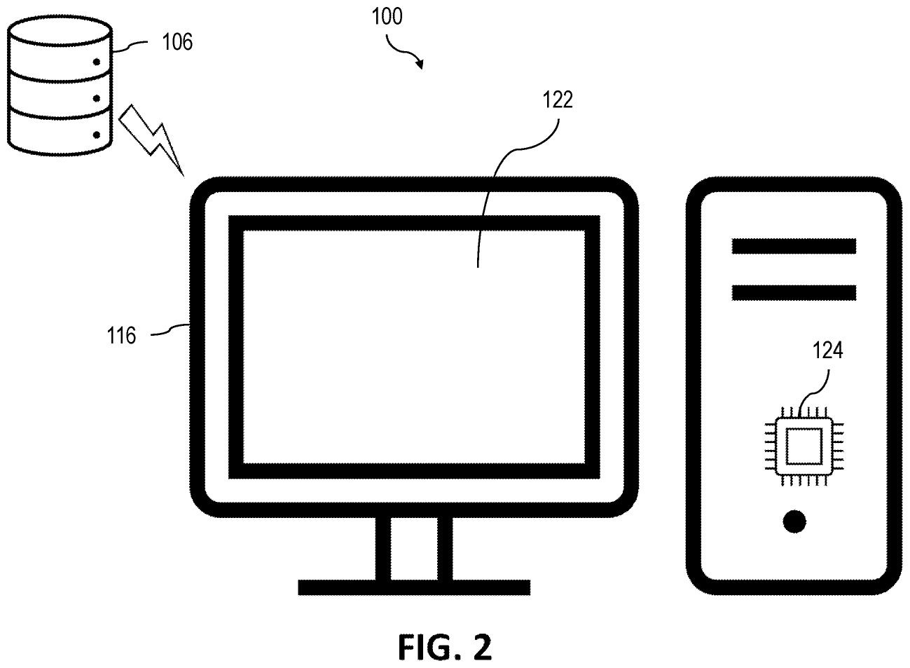

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the report platform 112 hosted by the server 106. The UI 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the UI 122 to log into the report platform 112 via a web browser such that the user can use and interact with the report platform 112.

### Machine Learning Models

The server 106 applies the one or more machine learning models 108A-C to the ECG data to analyze and classify the beats and cardiac activity of the patient 10.

The first and second machine learning models 108A and 108B are programmed to—among other things—compare the ECG data to labeled ECG data to determine which labeled ECG data the ECG data most closely resembles. The labeled ECG data may identify a particular cardiac event—including but not limited to ventricular tachycardia, bradycardia, atrial fibrillation, pause, normal sinus rhythm, or artifact/noise—as well as particular beat classifications—including but not limited to ventricular, normal, or supraventricular beats. In addition to identifying beat classifications and event classifications (and generating associated metadata), the first and second machine learning models 108A and 108B can determine and generate metadata regarding heart rates, duration, and beat counts of the patient 10 based on the ECG data. As specific examples, the first and/or the second machine learning models 108A and 108B can identify the beginning, center, and end of individual beats (e.g., individual T-waves) such that individual beats can be extracted from the ECG data. Each individual beat can be assigned a value (e.g., a unique identifier) such that individual beats can be identified and associated with metadata throughout processing and analyzing the ECG data.

The ECG data (e.g., ECG data associated with individual beats) as well as certain outputs of the first and second machine learning models 108A and 108B can be inputted to the third machine learning model 108C. Although two machine learning models are shown and described, a single machine learning model could be used to generate the metadata described herein, or additional machine learning models could be used.

The first and second machine learning models 108A and 108B can include the neural networks described in Ser. No. 16/695,534, which is hereby incorporated by reference in its entirety. The first neural network can be a deep convolutional neural network and the second neural network is a deep fully-connected neural network—although other types and combinations of machine learning models can be implemented. The first machine learning model 108A receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The second machine learning model 108B receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

The third machine learning model 108C (e.g., one or more trained encoder machine learning models) is programmed to generate latent space representations of the ECG data such that the ECG data is represented by fewer datapoints than the original ECG data. The latent space representations can be used as an approximation of the original raw ECG data for each beat. Although the inputs to the third machine learning model 108C are described as (1) the ECG data such as sets of individual T-waves and (2) certain outputs of the first and second machine learning models 108A and 108B, the third machine learning model 108C could be programmed to generate the latent space representations without requiring input from the first and/or second machine learning models 108A, 108B.

In certain instances, instead of a single third machine learning model 108C, the server 106 includes a separate machine learning model for each type of beat classification (e.g., normal beats, ventricular beats, and supraventricular beats). For example, as shown in FIG. 1, the server 106 may include three third machine learning models (108C-N, 108C-V, and 108C-S) instead of a single third machine learning model. In certain instances, beats that were not initially classified (e.g., unclassified beats) can be processed either by a different third machine learning model or can skip the step of generating latent space representations and being clustered with similar shaped beats.

In the example of FIG. 1, one machine learning model 108C-N is used for beats classified as normal beats, another machine learning model 108C-V is used for beats classified as ventricular beats, and another machine learning model 1080-S is used for beats classified as supraventricular beats. As such, only ECG data (e.g., T-waves) of beats initially classified as normal beats by the first and/or second machine learning models 108A, 108B—as well as metadata generated by such machine learning models—are inputted to the machine learning model 108C-N, and so on. It has been found that using machine learning models trained to focus on analyzing only certain types of beats can improve performance of the third machine learning models compared to using a single third machine learning model. Further, processing the ECG data in parallel using three machine learning models can decrease the time needed to generate the latent space representations. In certain instances, a single study may contain hundreds of thousands to millions of individual beats.

Each third machine learning model (108C-N, 108C-V, 108C-S) receives ECG data associated with individual beats (e.g., an individual clip of ECG data for each beat) and generates latent space representations of such ECG data. For example, each individual beat is processed by one of the third machine learning models-depending on each individual beat's classification-such that the ECG data is distilled down to (or represented by) a small number of individual data points. Raw ECG data of an individual beat can include 500 or so datapoints, and each third machine learning model can distill the ECG data for a given beat into 4-16 datapoints. Put another way, each third machine learning model can generate latent space representations comprising 4-16 datapoints for a given beat. This range has been found to balance accuracy of beat representation and effectiveness of clustering (described further below). In certain instances, the latent space representations comprise 7, 8, or 9 (e.g., 7-9) datapoints for a given beat. The latent space representations comprise 1-2% of datapoints compared to the raw ECG data for each beat. Each latent space can be represented by a vector (e.g., a latent vector).

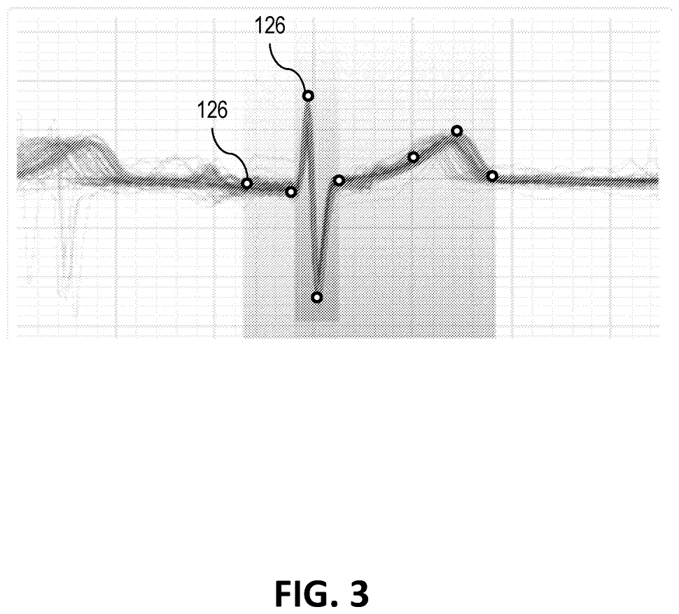

The resulting datapoints are representations of an amplitude of the ECG signal at different relative points in time. These limited datapoints are datapoints that the trained machine learning models generate such that different beat shapes can be identified and similar shaped beats can be grouped together. Put another way, these datapoints may be those that are the most likely to be helpful in distinguishing among beat shapes. The third machine learning models can leave out representations of datapoints that are less likely to help distinguish among individual beats. FIG. 3 shows an example set of beats that have been grouped or clustered together and also shows non-limiting examples of points 126 within a beat's ECG signal that may be useful for distinguishing among beat shapes. For example, the points 126 can be located at the beginning and end of each beat, apexes (e.g., QRS peak), nadirs, etc.

In the example of FIG. 1, the third machine learning models (108C-N, 108C-V, 108C-S) generate respective separate latent space representations for sets of beats initially classified as normal beats, ventricular beats, and supraventricular beats. In certain instances, beats that could not be initially classified (or ECG data containing artifacts due to noise) are not processed by any of the third machine learning models. Such beats can be labeled as unclassified beats.

The output(s) of the third machine learning model(s) 108C are processed by a clustering algorithm module 109. The clustering algorithm module 109 receives the latent space representations of individual beats and is programmed to associate similar shaped beats into different groups. FIG. 3 shows an example set of beats that have been grouped or clustered together. As shown in FIG. 3, ECG waveforms of individual beats (e.g., T-waves) are superimposed on each other. Each cluster or group can include hundreds or thousands of beats that have been grouped together by the clustering algorithm module 109. As can be seen, the beats all have a similar profile relative to each other. Each beat is aligned with the other beats to have respective QRS peaks centered on the graph.

In certain instances, the clustering algorithm module 109 is programmed to apply a clustering algorithm such as the k-means clustering algorithm or a derivation or variation thereof to the latent space representations. In certain instances, the same clustering algorithm module 109 and the same algorithm is used to process the latent space representations of each of the third machine learning models (108C-N, 108C-V, 108C-S). In certain instances, the output of the clustering algorithm module 109 includes assigning a value (e.g., an identifier such as a number) to each beat that is indicative of the group selected by the clustering algorithm module 109. For example, if the clustering algorithm module 109 clusters the beats into eight different groups, then all beats selected to be in the first group may be assigned a value of “1” and all beats selected to be in the second group may be assigned a value of “2” and so on. Other types of values can be used. These group values can be added to the metadata associated with each beat.

### Prioritizing Access to Ecg Data and Metadata

As previously noted, accessing and displaying days of ECG data can be an inefficient use of computing resources, network bandwidth resources, and human resources. To help address these resource challenges, the remote computer 116 and the server 106 can communicate with each other (e.g., via commands/requests and responses) to prioritize when and what ECG data and metadata are accessible to users at the remote computer 116.

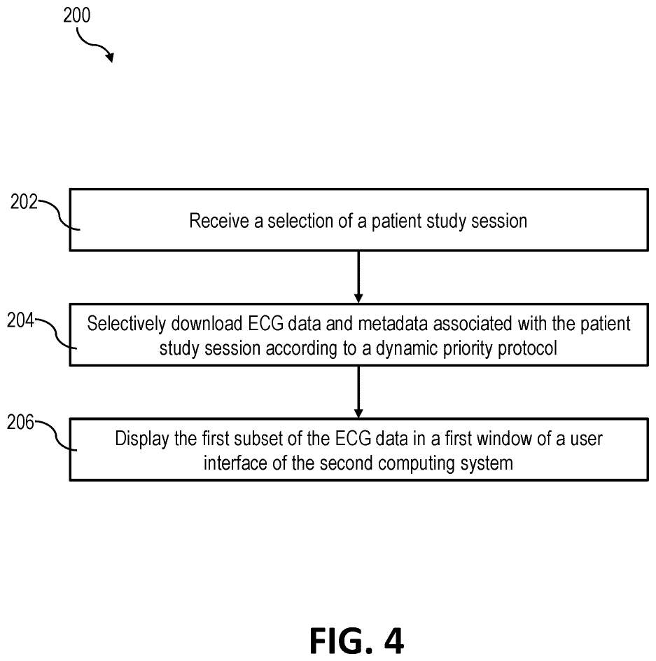

FIG. 4 outlines a method 200 for prioritizing access to ECG data and metadata. The prioritization can be carried out according to a dynamic priority protocol. In certain instances, the remote computer 116 operates the dynamic priority protocol and initiates commands/requests that are transmitted to the server 106, and the server 106 transmits ECG data and metadata in response to the commands/requests. For example, in some instances, the remote computer 116 receives executable code (e.g., JavaScript code) as part of an initial batch of files from the server 106, and the executable code includes code for carrying out the dynamic priority protocol. In another example, the server 106 stores and operates the code for carrying out the dynamic priority protocol.

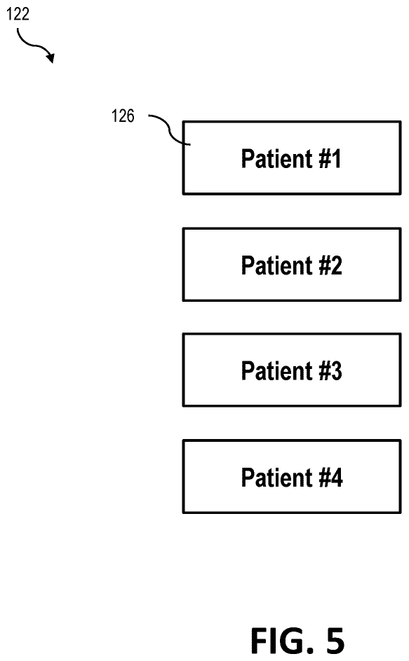

The method 200 includes receiving a selection of a patient study session (block 202 in FIG. 4). For example, a user at the remote computer 116 can initiate a patient study session by selecting an icon associated with a particular patient. FIG. 5 shows a schematic of the user interface 122 with a list of icons 126 that can be selected (e.g., via a mouse or keyboard) to initiate a patient study session.

Referring back to the method 200 of FIG. 4, once a patient study session is selected, the remote computer 116—in connection with the server 106—can begin to selectively download data associated with the patient study session from the server 106 to the remote computer 116 (block 204 in FIG. 4). The data can be downloaded in response to the remote computer 116 sending commands or requests to the server 106 for particular sets of data. The order of data being downloaded can be dictated by the dynamic priority protocol. In certain instances, the dynamic priority protocol can comprise a default set of priority rules, which can be dynamically adjusted based on a user's activity.

In certain instances, before raw ECG data is downloaded to the remote computer 116, the remote computer 116 sends a command or request to the server 106 for certain metadata. This metadata can include non-ECG patient data (e.g., name, physician) and pointers to ECG data and associated metadata. In certain instances, the pointers are used by the remote computer 116 to request specific strips of ECG data (e.g., specific time periods of ECG data) stored at the server 106 be downloaded to the remote computer 116. As such, to download particular strips of ECG data, the remote computer 116 can utilize the pointers to request the strips from the server 106.

The initial batch of metadata can be downloaded in a first data payload. Additionally or alternatively, the metadata initially downloaded can include beat data (e.g., classifications, locations in time, associated strip of ECG data) and cardiac event data (e.g., classifications, locations in time, associated strip of ECG data). This batch of metadata can be downloaded in a second data payload. Also, in certain instances, once a patient study session is selected, the remote computer 116 receives the executable code as described above. In other instances, the executable code is downloaded to the remote computer 116 when a user initially accesses the report platform 112 and before a patient study session is selected.

Once certain baseline metadata is downloaded, the dynamic priority protocol can prioritize how strips of raw ECG data from the patient study are downloaded to the remote computer 116. For example, instead of downloading raw ECG data chronologically, the dynamic priority protocol can dictate which selective portions of ECG data from the patient study are downloaded before other portions. Put another way, instead of starting with ECG data from the beginning of the study and downloading ECG data chronologically until completing with ECG data at the end of the study, selective portions of ECG data from the patient study can be requested by the remote computer 116 and downloaded regardless of where the portions are located in time. In particular, portions containing certain types of beats or rhythms can be prioritized over other portions.

As one example, the dynamic priority protocol can prioritize strips of ECG data that contain at least one beat that has been initially classified as an ectopic beat (e.g., a ventricular beat or a supraventricular beat). More specifically, the dynamic priority protocol can first prioritize strips of ECG data containing a ventricular beat and secondly prioritize strips of ECG data containing a supraventricular beat.

In certain instances, the raw ECG data of the patient study is split into many smaller chunks of ECG data. For example, the entire patient study may comprise separate individual strips of 5- to 10-minute intervals (or other time intervals) of ECG data. These individual strips of ECG data may be initially created by the monitor 102 and/or the mobile device 104 (of FIG. 1), which may be programmed to collect, store, and/or transmit predefined time intervals of ECG data. Further, the one or more machine learning models of the server 106 may be programmed to split and/or analyze smaller individual strips of ECG data. When the dynamic priority protocol prioritizes which strips of ECG data to download initially, individual strips of ECG data containing at least one ectopic beat (as noted above) can be prioritized first.

Referring back to the method 200 of FIG. 4, the strips of ECG data (or portions thereof) that have been downloaded to the remote computer 116 can be displayed on the user interface 122 (block 206 in FIG. 4). For example, plots of ECG data can be displayed in one or more windows of the user interface 122.

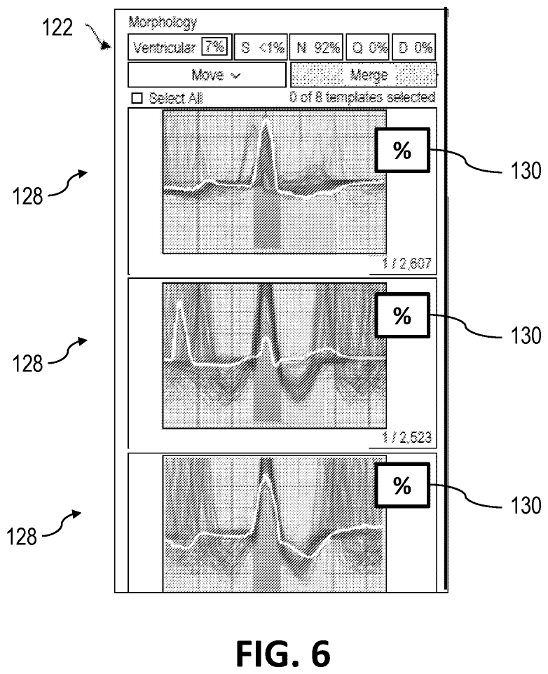

FIG. 6 shows a view of the user interface 122 with various windows 128. Each window 128 can display plots of beats that have the same initial beat classification. For example, one of the windows 128 can display plots of beats classified as ventricular beats, and another one of the windows 128 can display plots of beats classified as supraventricular beats, and so on. As can be seen, the plots of beats displayed in the windows have similar profiles relative to each other. Further, in the example in FIG. 6, each beat is aligned with the other beats to have respective QRS peaks centered within the windows 128, and the plots are superimposed over each other.

When prioritizing what strips of ECG data to download to the remote computer 116, the dynamic priority protocol can prioritize strips of ECG data that will have plots displayed in one of the windows 128 of the user interface 122. More specifically, the dynamic priority protocol can (1) prioritize strips of ECG data containing a ventricular beat that will be initially displayed in one of the windows 128, (2) then prioritize strips of ECG data containing a supraventricular beat that will initially be displayed in one of the windows 128, (3) then prioritize strips of ECG data containing a ventricular beat that will not be initially displayed in one of the windows 128, and (4) then prioritize strips of ECG data containing a supraventricular beat that will not be initially displayed in one of the windows 128.

As shown in FIG. 6, each window 128 can include an indicator 130 that displays a numerical value that indicates how many plots of ECG data (e.g., a percentage, a number of beats) that are associated with that window 128 have been downloaded already to the remote computer 116 or that remain to be downloaded.

As noted above, the dynamic priority protocol can comprise a default set of priority rules, which can be dynamically adjusted based on a user's activity. One default set of priority rules can involve prioritizing downloading strips of ECG data that contain one or more ectopic beats. Another default rule can involve prioritizing strips of ECG data that include a plot of a beat that will initially be displayed on the user interface 112. However, the default rules can be overridden by a user's activity.

As one example, when a user selects one of the indicators 130 displayed on the user interface 122, the dynamic priority protocol can prioritize downloading strips of ECG data that contain one or more beats associated with the group of beats with the window 128.

As another example, when a user scrolls through sections of the user interface 122 that should display ECG which has not been downloaded, the dynamic priority protocol can prioritize downloading strips of ECG data that are located at or near where the user is scrolling.

To facilitate prioritization of downloading raw ECG data, the dynamic priority protocol can utilize priority levels that are adjustable. For example, each strip of ECG data can be associated with its own priority level. In certain instances, this involves using a high priority level, a middle priority level, and a low priority level—although additional or fewer separate priority levels can be used. The high priority level can be initially populated by pointers (or other indicators) associated with strips of ECG data that contain beats with the various characteristics described above-such as ventricular beats for initial display. Once strips of ECG data containing such beats have been downloaded, the next highest priority strips of ECG can be downloaded. In the event that a user's activity overrides the default priority, the strips of ECG affected by the user's activity can be placed in the high priority level (or at least a level higher than the initial priority level). Strips of ECG data can be downloaded according to the dynamic priority protocol (and its priority levels) until all ECG data associated with the patient study has been downloaded to the remote computer 116.

The downloaded strips of ECG data and metadata can be displayed in various ways on the user interface 122. The user can then analyze the ECG data and metadata, modify initial beat and rhythm classifications (if needed), and ultimately generate a report such as a Holter report. As the user modifies initial metadata such as beat and rhythm classifications, changes can be carried out on the remote computer 116—as opposed to sending large chunks of data back and forth between the server 106 and the remote computer 116. Once the user is satisfied with a report, a final report can be generated and sent to the patient's physician.

In certain instances, once the report is built and complete, the remote computer 116 can send any changes to the metadata (e.g., the beat classifications and the rhythm classifications) to the server 106 and its database. The server 106 can then replace the metadata initially created by the machine learning model (and saved to the database) with the metadata generated by the remote computer while the user was reviewing and editing the metadata. As such, if the ECG data and metadata need to be accessed again, the server's database has the most recent version of the metadata. Further, machine learning model may be further trained on the metadata generated by the user at the remote computer.

### Computing Devices and Systems

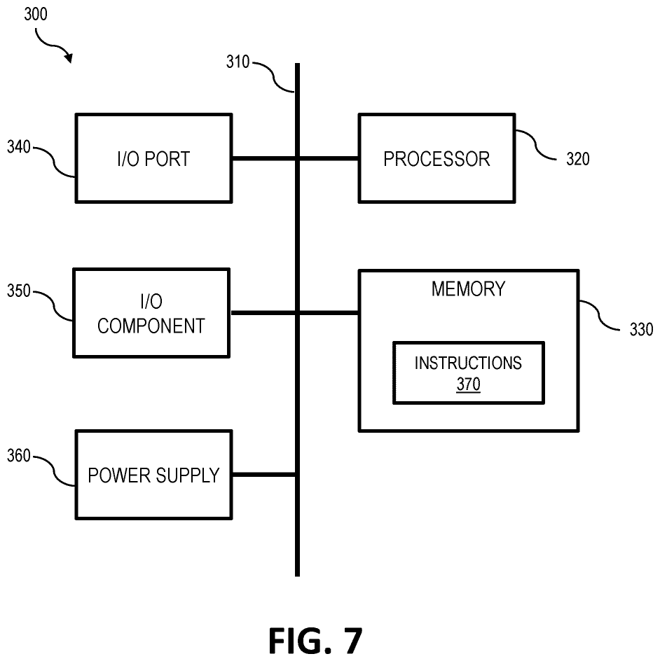

FIG. 7 is a block diagram depicting an illustrative computing device 300, in accordance with instances of the disclosure. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 7 and described below. For example, the monitor 102, the mobile device 104, the server 106, and the remote computer 116 can each include their own set of components shown in FIG. 7 and described below.

In instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a remote computing system comprising: a user interface, one or more processors, and a first computer-readable medium having a first set of computer-executable instructions configured to be executed to cause the remote computing system to:
send requests to a server to retrieve electrocardiogram (ECG) data associated with a patient study session, wherein the requests are generated according to a dynamic priority protocol, wherein the ECG data is associated with metadata, which includes beat classifications;
receive the ECG data in response to the requests; and
display, on the user interface, plots of the ECG data that have been received,
wherein the dynamic priority protocol prioritizes downloading strips of the ECG data associated with the beat classifications indicating ectopic beats.

2. The system of claim 1, wherein the beat classifications indicating ectopic beats are ventricular beats and supraventricular beats.

3. The system of claim 1, wherein the strips of ECG data containing at least one ventricular beat are prioritized first, wherein the strips of ECG data containing at least one supraventricular beat are prioritized second.

4. The system of claim 1, wherein the display of the plots of the ECG data includes: display the plots of the ECG data associated with ventricular beats in a first window in the user interface and displaying the plots of the ECG data associated with supraventricular beats in a second window in the user interface.

5. The system of claim 1, wherein the dynamic priority protocol is modified in response to detecting that a user is scrolling through sections of the user interface associated with ECG data that has not been downloaded.

6. The system of claim 1, wherein the first set of computer-executable instructions are further configured to be executed to cause the remote computing system to:
display on the user interface a first indicator representing an amount of the ECG data associated with a first beat classification that has been downloaded to the remote computer; and
display on the user interface a second indicator representing an amount of the ECG data associated with a second beat classification that has been downloaded to the remote computer.

7. The system of claim 6, wherein the ECG data associated with the first beat classification is prioritized for downloading in response to a user selecting the first indicator.

8. The system of claim 1, wherein the dynamic priority protocol includes a set of default rules.

9. The system of claim 8, wherein the default rules are modified by a user's activity on the user interface.

10. The system of claim 1, wherein the first set of instructions are part of the package of data received from the server.

11. The system of claim 10, wherein the first set of instructions are executed by a web browser at the remote computing system.

12. The system of claim 1, wherein the requests each include a pointer to respective strips of ECG data containing beat classifications indicating ectopic beats.

13. The system of claim 12, wherein the strips of ECG data are 5- to 10-minute-long strips.

14. The system of claim 1, wherein:
the server comprises: a database and a second computer-readable medium having a second set of computer-executable instructions configured to be executed to cause the server to:
in response to the requests, retrieve the ECG data from the database and transmit the strips of the ECG data to the remote computing system.

15. A method comprising:
receiving, at a remote computer, a selection of a patient study session;
selectively downloading, to the remote computer, electrocardiogram (ECG) data associated with the patient study session according to a dynamic priority protocol, wherein the ECG data is associated with metadata that includes beat classifications; and
displaying, on a user interface of the remote computer, plots of the ECG data that have been downloaded,
wherein the dynamic priority protocol prioritizes downloading strips of the ECG data associated with the beat classifications indicating ectopic beats.

16. The method of claim 15, wherein the beat classifications indicating ectopic beats are ventricular beats and supraventricular beats.

17. The method of claim 16, wherein the displaying includes: displaying the plots of the ECG data associated with ventricular beats in a first window in the user interface and displaying the plots of the ECG data associated with supraventricular beats in a second window in the user interface.

18. The method of claim 15, wherein the strips of ECG data containing at least one ventricular beat are prioritized first, wherein the strips of ECG data containing at least one supraventricular beat are prioritized second.

19. The method of claim 15, wherein the metadata with the beat classifications are downloaded to the remote computer before the ECG data is downloaded to the remote computer.

20. The method of claim 15, wherein the dynamic priority protocol includes a set of default rules, wherein the default rules are modified by a user's activity on the user interface.
